(19) United States
(12) Patent Application Publication
(10) Pub. No.: US 2026/0348964 A1
(43) Pub. Date: Nov. 5, 2026

(54) USE OF CELL FREE NUCLEOSOMES AS BIOMARKERS

(71) Applicant: Belgian Volition SRL, Isnes (BE)

(72) Inventors: Marielle HERZOG, Isnes (BE); Priscilla VAN DEN ACKERVEKEN, Isnes (BE); Jonathan DOUXFILS, Liége (BE)

(21) Appl. No.: 19/487,137

(22) PCT Filed: May 24, 2024
(86) PCT No.: PCT/EP2024/064401

§ 371 Date: Nov. 24, 2025

(30) Foreign Application Priority Data
May 24, 2023 (GB) 2307768.8

(51) Int. Cl. G01N 33/575; G01N 33/543; G01N 33/68

(52) U.S. Cl. CPC G01N 33/57557; G01N 33/54306; G01N 33/57585; G01N 33/57595; G01N 33/6875; G01N 33/6893

(57) ABSTRACT

The invention relates to methods of detecting or diagnosing brain tumours using cell free nucleosomes as biomarkers in a body fluid sample, as well as related uses and kits using said method.

### FIELD OF THE INVENTION

The present invention relates to cell free nucleosomes as biomarkers in a body fluid sample for a brain tumour.

### BACKGROUND OF THE INVENTION

Cancer is a common disease with a high mortality. The biology of the disease is understood to involve a progression from a pre-cancerous state leading to stage I, II, III and eventually stage IV cancer. For the majority of cancer diseases, mortality varies greatly depending on whether the disease is detected at an early localized stage, when effective treatment options are available, or at a late stage when the disease may have spread within the organ affected or beyond when treatment is more difficult.

High-grade malignant glioma, glioblastoma multiforme (GBM), is the most aggressive and lethal form of brain tumours with a survival rate less than 5% after 5 years. GBMs are a type of glioma (a brain tumour that grows from a glial cell). Glioblastomas often form from a type of glial cell called an astrocyte. The reference to ‘multiforme’ are due to the fact that glioblastomas have lots of variations, or different forms. This can unfortunately make them more difficult to treat than other tumours. Glioblastomas are categorised as grade 4 brain tumours.

Currently, glioblastoma (GBM) diagnosis and monitoring rely on neuroimaging and histopathological confirmation. However, overall survival has not improved in last decades due to therapeutic failure and to a lack of biomarkers for detection of relapse. Glioblastoma are generally diffuse which can make it difficult for the whole tumour to be removed through surgery. This makes monitoring methods essential to check patients recovering from glioblastoma do not relapse if some tumour tissue is left behind following surgery. Liquid biopsies (i.e. blood or cerebrospinal fluid) using nucleosomes-containing-histone post-translational modifications (PTMs) have the potential to become valuable biomarkers for diagnosis and monitoring GBM.

There is a need to develop non-invasive blood tests for detecting and monitoring patients with, or in remission from, a brain tumour, in particular as an adjunct to other cancer detection methods.

### BRIEF DESCRIPTION OF THE FIGURES

FIG. 1: The level of three candidate biomarkers compared to control cell lines (n=4) as measured by a blood-based immunoassay (Nu.Q®—commercially available from Volition): (A) H3K4Me4, (B) H3R8Cit and (C) H3S10Ph in GBM cell lines (n=4).

FIG. 2: The level of cell free nucleosomes comprising modifications H3K27Me3 (A) and H3K9Me3 (B) measured in GBM cell line SF-126 (n=3) with or without treatment of an EZH2 inhibitor, expressed as a ratio of total nucleosomes. The results are also confirmed by western blot analysis.

FIG. 3: Plasma samples taken from GMB patients and healthy patients. Nucleosomes were extracted and analysed for H3.1 in line with the methods described in Example 1. (A) The results are reported as ng/mL of the candidate biomarker. (B) ROC curves and AUCs were calculated for the discrimination of subjects with and without GMB for each assay using Logistic Regression analysis.

FIG. 4: Plasma samples taken from GMB patients and healthy patients. Nucleosomes were extracted and analysed for H3R8Cit in line with the methods described in Example 1. (A) The results are reported as ng/mL of the candidate biomarker. (B) ROC curves and AUCs were calculated for the discrimination of subjects with and without GMB for each assay using Logistic Regression analysis.

FIG. 5: Plasma samples taken from GMB patients and healthy patients. Nucleosomes were extracted and analysed for H3K4Me2 in line with the methods described in Example 1. (A) The results are reported as ng/mL of the candidate biomarker. (B) ROC curves and AUCs were calculated for the discrimination of subjects with and without GMB for each assay using Logistic Regression analysis.

### SUMMARY OF THE INVENTION

According to a first aspect, there is provided a use of a cell free nucleosome as a biomarker in a blood, serum or plasma sample, for the diagnosis or detecting of a brain tumour.

According to a further aspect of the invention, there is provided a use of a cell free nucleosome as a biomarker in a body fluid sample, for the diagnosis or detection of a glioblastoma.

According to a further aspect of the invention, there is provided a use of a panel of biomarkers in a body fluid sample for diagnosing and/or monitoring a brain tumour, wherein the biomarkers comprise H3R8Cit, H3S10Ph and H3K4Me2.

According to a further aspect of the invention, there is provided a method for diagnosing or detecting a brain tumour, which comprises the steps of:
(i) contacting a blood, serum or plasma sample obtained from the subject with a binding agent to detect or measure the level of cell free nucleosomes or a component thereof; and
(ii) using the level of cell free nucleosomes detected to diagnose the subject with the brain tumour.

According to a further aspect of the invention, there is provided a method for determining the prognosis of a subject with a brain tumour, which comprises the steps of:
(i) contacting a blood, serum or plasma sample obtained from the subject with a binding agent to detect or measure the level of cell free nucleosomes or a component thereof; and
(ii) using the level of cell free nucleosomes detected as indicative of the prognosis of said brain tumour.

According to a further aspect of the invention, there is provided a method for monitoring the efficacy of a therapy in a subject having, suspected of having, or being predisposed to a brain tumour, which comprises the steps of:
(i) contacting a blood, serum or plasma sample obtained from the subject with a binding agent to detect or measure the level of cell free nucleosomes or a component thereof; and
(ii) comparing the level of cell free nucleosomes detected with an earlier blood, serum or plasma sample taken from said subject to determine the efficacy of said therapy.

According to a further aspect of the invention, there is provided a method for diagnosing or detecting a glioblastoma, which comprises the steps of:
(i) contacting a body fluid sample obtained from the subject with a binding agent to detect or measure the level of cell free nucleosomes or a component thereof; and
(ii) using the level of cell free nucleosomes detected to diagnose the subject with the glioblastoma.

According to a further aspect of the invention, there is provided a method for determining the prognosis of a subject with a glioblastoma, which comprises the steps of:
(i) contacting a body fluid sample obtained from the subject with a binding agent to detect or measure the level of cell free nucleosomes or a component thereof; and
(ii) using the level of cell free nucleosomes detected as indicative of the prognosis of said glioblastoma.

According to a further aspect of the invention, there is provided a method for monitoring the efficacy of a therapy in a subject having, suspected of having, or being predisposed to a glioblastoma, which comprises the steps of:
(i) contacting a body fluid sample obtained from the subject with a binding agent to detect or measure the level of cell free nucleosomes or a component thereof; and
(ii) comparing the level of cell free nucleosomes detected with an earlier body fluid sample taken from said subject to determine the efficacy of said therapy.

According to a further aspect of the invention, there is provided a kit comprising one or more reagents for carrying out the method as defined herein.

According to a further aspect of the invention, there is provided a kit comprising one or more reagents to detect H3R8Cit, H3S10Ph and H3K4Me2 in a body fluid sample.

According to a further aspect of the invention, there is provided a kit comprising one or more reagents to detect H3.1, H3R8Cit and H3K4Me2 in a body fluid sample.

### DETAILED DESCRIPTION

According to a first aspect, there is provided a use of a cell free nucleosome as a biomarker in a blood, serum or plasma sample, for the diagnosis or detection of a brain tumour. In particular, the brain tumour is glioblastoma.

According to a further aspect of the invention, there is provided a use of a cell free nucleosome as a biomarker in a body fluid sample, for the diagnosis or detection of a glioblastoma. The nucleosome is the basic unit of chromatin structure and consists of a protein complex of eight highly conserved core histones (comprising of a pair of each of the histones H2A, H2B, H3, and H4). Around this complex is wrapped approximately 146 base pairs of DNA. Another histone, H1 or H5, acts as a linker and is involved in chromatin compaction. The DNA is wound around consecutive nucleosomes in a structure often said to resemble “beads on a string” and this forms the basic structure of open or euchromatin. In compacted or heterochromatin this string is coiled and super coiled into a closed and complex structure (Herranz and Esteller (2007) Methods Mol. Biol. 361: 25-62).

References to “nucleosome” may refer to “cell free nucleosome” when detected in body fluid samples. It will be appreciated that the term cell free nucleosome throughout this document is intended to include any cell free chromatin fragment that includes one or more nucleosomes. “Epigenetic features”, “epigenetic signal features” or “epigenetic signal structures” of a cell free nucleosome as referred herein may comprise, without limitation, one or more histone post-translational modifications, histone isoforms, modified nucleotides and/or proteins bound to a nucleosome in a nucleosome-protein adduct.

It will be understood that the cell free nucleosome may be detected by binding to a component thereof. The term “component thereof” as used herein refers to a part of the nucleosome, i.e. the whole nucleosome does not need to be detected. The component of the cell free nucleosomes may be selected from the group consisting of: a histone protein (i.e. histone H1, H2A, H2B, H3 or H4), a histone post-translational modification, a histone variant or isoform, a protein bound to the nucleosome (i.e. a nucleosome-protein adduct), a DNA fragment associated with the nucleosome and/or a modified nucleotide associated with the nucleosome. For example, the component thereof may be histone (isoform) H3.1 or histone H1 or DNA.

Methods and uses of the invention may measure the level of (cell free) nucleosomes per se. References to “nucleosomes per se” refers to the total nucleosome level or concentration present in the sample, regardless of any epigenetic features the nucleosomes may or may not include. Detection of the total nucleosome level typically involves detecting a histone protein common to all nucleosomes, such as histone H4. Therefore, nucleosomes per se may be measured by detecting a core histone protein, such as histone H4. As described herein, histone proteins form structural units known as nucleosomes which are used to package DNA in eukaryotic cells. As previously reported in WO 2016/067029 (incorporated herein by reference), particular histone variants, such as histone H3.1, H3.2 or H3t, may be used to improve the detection of cell free nucleosomes originating from tumour cells. Therefore, the total level of cell free nucleosomes of tumour origin may be detected.

Normal cell turnover in adult humans involves the creation by cell division of some 1011 cells daily and the death of a similar number, mainly by apoptosis. During the process of apoptosis chromatin is broken down into mononucleosomes and oligonucleosomes which are released from the cells. Under normal conditions the levels of circulating nucleosomes found in healthy subjects is reported to be low. Elevated levels are found in subjects with a variety of conditions including many cancers, auto-immune diseases, inflammatory conditions, stroke and myocardial infarction (Holdenrieder & Stieber (2009) Crit Rev Clin Lab Sci, 46(1): 1-24).

Current nucleosome ELISA methods are used primarily in cell culture, usually as a method to detect apoptosis (Salgame et al. (1997) Nucleic Acids Res, 25(3): 680-681; Holdenrieder et al. (2001) supra; van Nieuwenhuijze et al. (2003) Ann Rheum Dis, 62: 10-14), but are also used for the measurement of circulating cell free nucleosomes in serum and plasma (Holdenrieder et al. (2001)). Cell free serum and plasma nucleosome levels released into the circulation by dying cells have been measured by ELISA methods in studies of a number of different cancers to evaluate their use as a potential biomarker. Mean circulating nucleosome levels are reported to be high in most, but not all, cancers studied. However, patients with malignant tumours are reported to have serum nucleosome concentrations that varied considerably and some patients with advanced tumour disease were found to have low circulating nucleosome levels, within the range measured for healthy subjects (Holdenrieder et al. (2001)).

The cell free nucleosome may be a mononucleosome or oligonucleosome, or a mixture thereof.

Mononucleosomes and oligonucleosomes can be detected by Enzyme-Linked ImmunoSorbant Assay (ELISA) and several methods have been reported (e.g. Salgame et al. (1997); Holdenrieder et al. (2001); van Nieuwenhuijze et al. (2003)). These assays typically employ an anti-histone antibody (for example anti-H2B, anti-H3 or anti-H1, H2A, H2B, H3 and H4) as capture antibody and an anti-DNA or anti-H2A-H2B-DNA complex antibody as detection antibody.

Circulating nucleosomes are not a homogeneous group of protein-nucleic acid complexes. Rather, they are a heterogeneous group of chromatin fragments originating from the digestion of chromatin on cell death and include an immense variety of epigenetic structures including particular histone isoforms (or variants), post-translational histone modifications, nucleotides or modified nucleotides, and protein adducts. It will be clear to those skilled in the art that an elevation in nucleosome levels will be associated with elevations in some circulating nucleosome subsets containing particular epigenetic signals including nucleosomes comprising particular histone isoforms (or variants), comprising particular post-translational histone modifications, comprising particular nucleotides or modified nucleotides and comprising particular protein adducts. Assays for these types of chromatin fragments are known in the art (for example, see WO 2005/019826, WO 2013/030579, WO 2013/030578, WO 2013/084002 which are herein incorporated by reference).

The biomarker used in the uses and methods of the invention may be the level of cell free nucleosomes per se and/or an epigenetic feature of a cell free nucleosome. It will be understood that the terms “epigenetic signal structure” and “epigenetic feature” are used interchangeably herein. They refer to particular features of the nucleosome that may be detected. In one embodiment, the epigenetic feature of the nucleosome is selected from the group consisting of: a post-translational histone modification, a histone variant, a particular nucleotide and a protein adduct.

In one embodiment, the epigenetic feature of the nucleosome comprises one or more histone variants or isoforms. The epigenetic feature of the cell free nucleosome may be a histone isoform, such as a histone isoform of a core nucleosome, in particular a histone H3 isoform. The term “histone variant” and “histone isoform” may be used interchangeably herein. The structure of the nucleosome can also vary by the inclusion of alternative histone isoforms or variants which are different gene or splice products and have different amino acid sequences. Many histone isoforms are known in the art. Histone variants can be classed into a number of families which are subdivided into individual types. The nucleotide sequences of a large number of histone variants are known and publicly available for example in the National Human Genome Research Institute NHGRI Histone Database (Mariño-Ramírez et al. The Histone Database: an integrated resource for histones and histone fold-containing proteins. Database Vol.2011. and http://genome.nhgri.nih.gov/histones/complete.shtml), the GenBank (NIH genetic sequence) Database, the EMBL Nucleotide Sequence Database and the DNA Data Bank of Japan (DDBJ). For example, variants of histone H2 include H2A1, H2A2, mH2A1, mH2A2, H2AX and H2AZ. In another example, histone isoforms of H3 include H3.1, H3.2 and H3t.

In one embodiment, the histone isoform is H3.1.

The structure of nucleosomes can vary by post translational modification (PTM) of histone proteins. PTM of histone proteins typically occurs on the tails of the core histones and common modifications include acetylation, methylation or ubiquitination of lysine residues as well as methylation of arginine residues and phosphorylation of serine residues and many others. Many histone modifications are known in the art and the number is increasing as new modifications are identified (Zhao and Garcia, 2015 Cold Spring Harb Perspect Biol, 7: a025064). Therefore, in one embodiment, the epigenetic feature of the cell free nucleosome may be a histone post translational modification (PTM). The histone PTM may be present on a core nucleosome histone (e.g. H2A, H2B, H3 or H4), or a linker histone (e.g. H1 or H5). The histone PTM may be a histone PTM of a core nucleosome, e.g. H3, H2A, H2B or H4, in particular H3, H2A or H2B. In particular, the histone PTM is a histone H3 PTM. Examples of such PTMs are described in WO 2005/019826 and WO 2017/068359.

For example, the post translational modification may include acetylation, methylation, which may be mono-, di-or tri-methylation, phosphorylation, ribosylation, citrullination, ubiquitination, hydroxylation, glycosylation, nitrosylation, glutamination and/or isomerisation (see Ausio (2001) Biochem Cell Bio 79: 693). In one embodiment, the histone PTM is methylation of a lysine residue. In a further embodiment, the methylation is of a histone 3 lysine residue. In a yet further embodiment, the histone PTM is selected from H3K4Me, H3K4Me2, H3K9Me, H3K9Me3, H3K27Me3 or H3K36Me3. In one embodiment, the histone PTM is acetylation of a lysine residue. In a further embodiment, the acetylation is of a histone 3 lysine residue. In a yet further embodiment, the histone PTM is selected from H3K9Ac, H3K14Ac, H3K18Ac or H3K27Ac. In another embodiment, the histone PTM is H4PanAc. In one embodiment, the histone PTM is phosphorylation of a serine residue. In a further embodiment, the phosphorylation is of an isoform X of histone 2A (H2AX) serine residue or phosphorylation of a histone 3 serine residue. In a yet further embodiment, the histone PTM is selected from pH2AX or H3S10Ph. In one embodiment, the histone PTM is selected from citrullination or ribosylation. In a further embodiment, the histone PTM is citrullinated H3 (H3cit) or citrullinated H4 (H4cit). In a further embodiment, the histone PTM is citrullination of a histone 3 arginine residue. In a yet further embodiment, the histone PTM is H3R8Cit. In one embodiment, the histone PTM is selected from the group consisting of: H3K4Me, H3K4Me2, H3K9Me, H3K9Me3, H3K27Me3, H3K36Me3, H3K9Ac, H3K14Ac, H3K18Ac, H3K27Ac, H4PanAc, pH2AX, H3S10Ph and H3R8Cit.

In one embodiment, the histone PTM is selected from one or more of: H3R8Cit, H3S10Ph and H3K4Me2.

In one embodiment, the histone PTM is selected from one or more of: H3R8Cit and H3K4Me2.

In another embodiment, the histone PTM is selected from H3R8Cit and H3S10Ph.

A group or class of related histone post translational modifications (rather than a single modification) may also be detected. A typical example, without limitation, would involve a 2-site immunoassay employing one antibody or other selective binder directed to bind to nucleosomes and one antibody or other selective binder directed to bind the group of histone modifications in question. Examples of such antibodies directed to bind to a group of histone modifications would include, for illustrative purposes without limitation, anti-pan-acetylation antibodies (e.g. a Pan-acetyl H4 antibody [H4panAc]), anti-citrullination antibodies or anti-ubiquitin antibodies. In one embodiment, the histone PTM is selected from citrullination or ribosylation, in particular citrullination. In a further embodiment, the histone PTM is H3 citrulline (H3cit) or H4 citrulline (H4cit). In a yet further embodiment, the histone PTM is H3cit.

In one embodiment, the epigenetic feature of the nucleosome comprises one or more DNA modifications. In addition to the epigenetic signalling mediated by nucleosome histone isoform and PTM composition, nucleosomes also differ in their nucleotide and modified nucleotide composition. Global DNA hypomethylation is a hallmark of cancer cells and some nucleosomes may comprise more 5-methylcytosine residues (or 5-hydroxymethylcytosine residues or other nucleotides or modified nucleotides) than other nucleosomes. In one embodiment, the DNA modification is selected from 5-methylcytosine or 5-hydroxymethylcytosine.

In one embodiment, the epigenetic feature of the nucleosome comprises one or more protein-nucleosome adducts or complexes. A further type of circulating nucleosome subset is nucleosome protein adducts. It has been known for many years that chromatin comprises a large number of non-histone proteins bound to its constituent DNA and/or histones. These chromatin associated proteins are of a wide variety of types and have a variety of functions including transcription factors, transcription enhancement factors, transcription repression factors, histone modifying enzymes, DNA damage repair proteins and many more. These chromatin fragments including nucleosomes and other non-histone chromatin proteins or DNA and other non-histone chromatin proteins are described in the art.

In one embodiment, the protein adducted to the nucleosome (and which therefore may be used as a biomarker) is selected from: a transcription factor, a High Mobility Group Protein or chromatin modifying enzyme. References to “transcription factor” refer to proteins that bind to DNA and regulate gene expression by promoting (i.e. activators) or suppressing (i.e. repressors) transcription. Transcription factors contain one or more DNA-binding domains (DBDs), which attach to specific sequences of DNA adjacent to the genes that they regulate. All of the circulating nucleosomes and nucleosome moieties, types or subgroups described herein may be useful in the present invention.

Another way the structure of nucleosomes may vary is by mutation. Therefore, in one embodiment, the epigenetic feature is a mutated histone. In a further embodiment, the mutation is in histone 3 (H3). In a yet further embodiment, the mutation in H3 is when lysine 27 is replaced by a methionine (H3K27M).

It will be understood that more than one epigenetic feature of cell free nucleosomes may be detected in methods and uses of the invention. Multiple biomarkers may be used as a combined biomarker. Therefore, in one embodiment, the use comprises more than one epigenetic feature of cell free nucleosomes as a combined biomarker. The epigenetic features may be the same type (e.g. PTMs, histone isoforms, nucleotides or protein adducts) or different types (e.g. a PTM in combination with a histone isoform). For example, a post-translational histone modification and a histone variant may be detected (i.e. more than one type of epigenetic feature is detected). Alternatively, or additionally, more than one type of post-translational histone modification is detected, or more than one type of histone isoform is detected. In one aspect, the use comprises a post-translational histone modification and a histone isoform as a combined biomarker in a body fluid sample, for the diagnosis or detection of a brain tumour.

The panel of biomarkers may comprise the histone post-translational modifications: H3R8Cit (i.e. citrullinated histone H3 at the arginine residue in position 8), H3S10Ph (i.e. phosphorylated histone H3 at the serine residue in position 10), and H3K4Me2 (i.e. di-methylated histone H3 at the lysine residue in position 4). Therefore, according to one aspect of the invention, there is provided the use of a panel of biomarkers in a body fluid sample for diagnosing and/or monitoring a brain tumour, wherein the biomarkers comprise H3R8Cit, H3S10Ph and H3K4Me2.

The panel of biomarkers may comprise the histone isoform H3.1 and one or more of the histone post-translational modifications: H3R8Cit, H3S10Ph, and H3K4Me2. Therefore, according to one aspect of the invention, there is provided the use of a panel of biomarkers in a body fluid sample for diagnosing and/or monitoring a brain tumour, wherein the biomarkers comprise H3.1, H3R8Cit and H3K4Me2 or H3.1 and H3R8Cit.

The term “biomarker” means a distinctive biological or biologically derived indicator of a process, event, or condition. Biomarkers can be used in methods of diagnosis, e.g. clinical screening, and prognosis assessment and in monitoring the results of therapy, identifying patients most likely to respond to a particular therapeutic treatment, drug screening and development. Biomarkers and uses thereof are valuable for identification of new drug treatments and for discovery of new targets for drug treatment.

The sample may be any biological fluid (or body fluid) sample taken from a subject including, without limitation, cerebrospinal fluid (CSF), whole blood, blood serum, plasma, menstrual blood, endometrial fluid, urine, saliva, or other bodily fluid (stool, tear fluid, synovial fluid, sputum), breath, e.g. as condensed breath, or an extract or purification therefrom, or dilution thereof. In a preferred embodiment, the body fluid sample is selected from blood, serum or plasma. Biological samples also include specimens from a live subject, or taken post-mortem. The samples can be prepared, for example where appropriate diluted or concentrated, and stored in the usual manner.

It will be understood that methods and uses of the present invention find particular use in blood, serum or plasma samples obtained from a patient. In one embodiment, the sample is a blood or plasma sample. In a further embodiment, the sample is a serum sample. In a further embodiment both serum and plasma samples are used for the measurement of different members of an assay panel.

Preferably, plasma samples are used. Plasma samples may be collected in collection tubes containing one or more anticoagulants such as ethylenediamine tetraacetic acid (EDTA), heparin, or sodium citrate, in particular EDTA.

### Brain Tumours

The present invention is applicable to brain tumours.

In a further embodiment, the brain tumour is selected from glioma (such as glioblastoma multiforme, oligodendroglioma, ependymomas, brain stem glioma), craniopharyngioma, haemangioblastoma, malignant meningioma, pineal region tumours and vestibular schwannoma. In a yet further embodiment, the brain cancer is glioblastoma, in particular glioblastoma multiforme.

High-grade malignant glioma, glioblastoma multiforme (GBM), is the most aggressive and lethal form of brain tumours with a survival rate less than 5% after 5 years. GBMs are a type of glioma (a brain tumour that grows from a glial cell). Glioblastomas form from a type of glial cell called an astrocyte. The reference to ‘multiforme’ are due to the fact that glioblastomas have lots of variations, or different forms. This can unfortunately make them more difficult to treat than other tumours. Glioblastomas are categorised as grade 4 brain tumours.

In one embodiment, the brain tumour is a primary tumour.

### Detection and Diagnosis Methods

The invention provides methods which can be used in the detection or diagnosis of patients with a brain tumour. Therefore, according to a further aspect, there is provided a method for diagnosing or detecting a brain tumour which comprises the steps of:
(i) contacting a blood, serum or plasma sample obtained from the subject with a binding agent to detect or measure cell free nucleosomes; and
(ii) using the level of cell free nucleosomes detected to diagnose the subject with the brain tumour.

According to a further aspect of the invention, there is provided a method for diagnosing or detecting a glioblastoma, which comprises the steps of:
(i) contacting a body fluid sample obtained from the subject with a binding agent to detect or measure the level of cell free nucleosomes or a component thereof; and
(ii) using the level of cell free nucleosomes detected to diagnose the subject with the glioblastoma.

According to a further aspect, there is provided a method for determining the prognosis of a subject with a brain tumour, which comprises the steps of:
(i) contacting a blood, serum or plasma sample obtained from the subject with a binding agent to detect or measure cell free nucleosomes; and
(ii) using the level of cell free nucleosomes detected as indicative of the prognosis of said brain tumour.

According to a further aspect of the invention, there is provided a method for determining the prognosis of a subject with a glioblastoma, which comprises the steps of:
(i) contacting a body fluid sample obtained from the subject with a binding agent to detect or measure the level of cell free nucleosomes or a component thereof; and
(ii) using the level of cell free nucleosomes detected as indicative of the prognosis of said glioblastoma.

If a subject is determined to not have a brain tumour, then the invention may still be used for the purposes of monitoring disease progression. For example, if the use comprises a sample from a subject determined not to have a brain tumour, then the biomarker level measurements can be repeated at another time point to establish if the biomarker level has changed.

According to a further aspect, there is provided a method for monitoring the efficacy of a therapy in a subject having, suspected of having, or being predisposed to a brain tumour, which comprises the steps of:
(i) contacting a blood, serum or plasma sample obtained from the subject with a binding agent to detect or measure cell free nucleosomes; and
(ii) comparing the level of cell free nucleosomes detected with an earlier blood, serum or plasma sample taken from said subject to determine the efficacy of said therapy.

According to a further aspect of the invention, there is provided a method for monitoring the efficacy of a therapy in a subject having, suspected of having, or being predisposed to a glioblastoma, which comprises the steps of:
(i) contacting a body fluid sample obtained from the subject with a binding agent to detect or measure the level of cell free nucleosomes or a component thereof; and
(ii) comparing the level of cell free nucleosomes detected with an earlier body fluid sample taken from said subject to determine the efficacy of said therapy.

Detecting and/or quantifying may be performed directly on the purified or enriched nucleosome sample, or indirectly on an extract therefrom, or on a dilution thereof. Quantifying the amount of the biomarker present in a sample may include determining the concentration of the biomarker present in the sample.

Quantifying the level of cell free nucleosomes associated with an epigenetic epitope (in particular a histone post-translational modification, such as H3R8Cit, H3S10Ph and/or H3K4Me2) may be normalised on quantification of total nucleosomes, or the level of nucleosomes associated with a particular histone, such as histone isoform H3.1 or the level of DNA. References herein are made to “H3.1-nucleosomes” when referring to the level of nucleosomes containing histone isoform H3.1. Therefore, in one embodiment, the level of cell free nucleosome biomarker detected or measured, as described herein, is normalised against the level of cell free nucleosomes or a component thereof, in particular H3.1-nucleosomes.

Uses and methods of detecting, monitoring and of diagnosis according to the invention described herein are useful to confirm the existence of a disease, to monitor development of the disease by assessing onset and progression, or to assess amelioration or regression of the disease. Uses and methods of detecting, monitoring and of diagnosis are also useful in methods for assessment of clinical screening, prognosis, choice of therapy, evaluation of therapeutic benefit, i.e. for drug screening and drug development.

The detection or measurement may comprise an immunoassay, immunochemical, mass spectroscopy, chromatographic, chromatin immunoprecipitation or biosensor method. In particular, detection and/or measurement may comprise a 2-site immunoassay method for nucleosome moieties. Such a method is preferred for the measurement of nucleosomes or nucleosome incorporated epigenetic features in situ employing two anti-nucleosome binding agents or an anti-nucleosome binding agent in combination with an anti-histone modification or anti-histone variant or anti-DNA modification or anti-adducted protein detection binding agent. Also, detection and/or measurement may comprise a 2-site immunoassay employing a labelled anti-nucleosome detection binding agent in combination with an immobilized anti-histone modification or anti-histone variant or anti-DNA modification or anti-adducted protein binding agent.

The inventors herein used a 2-site immunoassay for detecting H3.1-nucleosomes by employing an immobilized anti-histone H3.1 antibody directed to bind to an epitope around amino acids 30-33 of the histone H3.1 protein to capture clipped and non-clipped nucleosomes, together with a labelled anti-nucleosome antibody directed to bind to an epitope present in intact nucleosomes but not present on isolated (free) histone or DNA nucleosome components. This type of epitope may be referred to as a “conformational nucleosome epitope” herein because it requires the native three-dimensional configuration of the target nucleosome to be intact.

In one embodiment, the method of detection or measurement comprises contacting the body fluid sample with a solid phase comprising a binding agent that detects cell free nucleosomes or a component thereof, and detecting binding to said binding agent.

In one embodiment, the method of detection or measurement comprises: (a) contacting the sample with a first binding agent which binds to an epigenetic feature of a cell free nucleosome; (b) contacting the sample bound by the first binding agent in step (a) with a second binding agent which binds to cell free nucleosomes; and (c) detecting or quantifying the binding of the second binding agent in the sample.

In another embodiment, the method of detection or measurement comprises: (a) contacting the sample with a first binding agent which binds to cell free nucleosomes; (b) contacting the sample bound by the first binding agent in step (a) with a second binding agent which binds to an epigenetic feature of the cell free nucleosome; and (c) detecting or quantifying the binding of the second binding agent in the sample.

Detecting or measuring the level of the biomarker(s) may be performed using one or more reagents, such as a suitable binding agent. For example, the one or more binding agents may comprise a ligand or binder specific for the desired biomarker, e.g. nucleosomes or component part thereof, an epigenetic feature of a nucleosome, a structural/shape mimic of the nucleosome or component part thereof, optionally in combination with one or more interleukins.

It will be clear to those skilled in the art that the terms “antibody”, “binder” or “ligand” as used herein are not limiting but are intended to include any binder capable of binding to particular molecules or entities and that any suitable binder can be used in the method of the invention. It will also be clear that the term “nucleosomes” is intended to include mononucleosomes and oligonucleosomes and any protein-DNA chromatin fragments that can be analysed in fluid media. In one embodiment, the binding agent, such as the antibody, specifically binds to the target biomarker. The specificity of an antibody is the ability of the antibody to recognize a particular antigen as a unique molecular entity and distinguish it from another. An antibody that “specifically binds” to an antigen or an epitope is a term well understood in the art. A molecule is said to exhibit “specific binding” if it reacts more frequently, more rapidly, with greater duration and/or with greater affinity with a particular target antigen or epitope, than it does with alternative targets. An antibody “specifically binds” to a target antigen or epitope if it binds with greater affinity, avidity, more readily, and/or with greater duration than it binds to other substances.

Methods of detecting biomarkers are known in the art. The reagents may comprise one or more ligands or binders, for example, naturally occurring or chemically synthesised compounds, capable of specific binding to the desired target. A ligand or binder may comprise a peptide, an antibody or a fragment thereof, or a synthetic ligand such as a plastic antibody, or an aptamer or oligonucleotide, capable of specific binding to the desired target. The antibody can be a monoclonal antibody or a fragment thereof. It will be understood that if an antibody fragment is used then it retains the ability to bind the biomarker so that the biomarker may be detected (in accordance with the present invention). A ligand/binder may be labelled with a detectable marker, such as a luminescent, fluorescent, enzyme or radioactive marker; alternatively or additionally a ligand according to the invention may be labelled with an affinity tag, e.g. a biotin, avidin, streptavidin or His (e.g. hexa-His) tag. Alternatively, ligand binding may be determined using a label-free technology for example that of ForteBio Inc.

The term “detecting” or “diagnosing” as used herein encompasses identification, confirmation, and/or characterisation of a disease state. Methods of detecting, monitoring and of diagnosis according to the invention are useful to confirm the existence of a disease, to monitor development of the disease by assessing onset and progression, or to assess amelioration or regression of the disease. Methods of detecting, monitoring and of diagnosis are also useful in methods for assessment of clinical screening, prognosis, choice of therapy, evaluation of therapeutic benefit, i.e. for drug screening and drug development.

In one embodiment, the method described herein is repeated on multiple occasions. This embodiment provides the advantage of allowing the detection results to be monitored over a time period. Such an arrangement will provide the benefit of monitoring or assessing the efficacy of treatment of a disease state. Such monitoring methods of the invention can be used to monitor onset, progression, stabilisation, amelioration, relapse and/or remission.

In monitoring methods, test samples may be taken on two or more occasions. The method may further comprise comparing the level of the biomarker(s) present in the test sample with one or more control(s) and/or with one or more previous test sample(s) taken earlier from the same test subject, e.g. prior to commencement of therapy, and/or from the same test subject at an earlier stage of therapy. The method may comprise detecting a change in the nature or amount of the biomarker(s) in test samples taken on different occasions.

A change in the level of the biomarker in the test sample relative to the level in a previous test sample taken earlier from the same test subject may be indicative of a beneficial effect, e.g. stabilisation or improvement, of said therapy on the disorder or suspected disorder. Furthermore, once treatment has been completed, the method of the invention may be periodically repeated in order to monitor for the recurrence of a disease.

Methods for monitoring efficacy of a therapy can be used to monitor the therapeutic effectiveness of existing therapies and new therapies in human subjects and in non-human animals (e.g. in animal models). These monitoring methods can be incorporated into screens for new drug substances and combinations of substances.

In a further embodiment the monitoring of more rapid changes due to fast acting therapies may be conducted at shorter intervals of hours or days.

According to a further aspect, there is provided a kit comprising one or more reagents for carrying out the method as defined herein. According to a further aspect, there is provide the use of a kit comprising one or more reagents to detect or measure the level of cell free nucleosomes or a component thereof, to detect, monitor or diagnose a brain tumour.

According to a further aspect of the invention, there is provided a kit to detect, monitor or diagnose a brain tumour in a subject, wherein said kit comprises a first binding agent which specifically binds to an epigenetic feature of a cell free nucleosome (e.g. H3.1) and a second binding agent which specifically binds to cell free nucleosomes.

According to a further aspect of the invention, there is provided a kit comprising one or more reagents to detect H3.1, H3R8Cit, H3S10Ph and H3K4Me2 in a body fluid sample. In one embodiment, the kit comprises one or more reagents to detect H3R8Cit, H3S10Ph and H3K4Me2 in a body fluid sample. In another embodiment, the kit comprises one or more reagents to detect H3.1, H3R8Cit and H3K4Me2 in a body fluid sample. In another embodiment, the kit comprises reagents to detect H3.1 and H3R8Cit.

Diagnostic or monitoring kits (or panels) are provided for performing methods of the invention. Such kits will suitably comprise one or more ligands for detection and/or quantification of the biomarker according to the invention, and/or a biosensor, and/or an array as described herein, optionally together with instructions for use of the kit.

A further aspect of the invention is a kit for detecting the presence of a disease state, comprising a biosensor capable of detecting and/or quantifying one or more of the biomarkers as defined herein. As used herein, the term “biosensor” means anything capable of detecting the presence of the biomarker. Examples of biosensors are described herein. Biosensors may comprise a ligand binder or ligands, as described herein, capable of specific binding to the biomarker. Such biosensors are useful in detecting and/or quantifying a biomarker of the invention.

Suitably, biosensors for detection of one or more biomarkers combine biomolecular recognition with appropriate means to convert detection of the presence, or quantitation, of the biomarker in the sample into a signal. Biosensors can be adapted for “alternate site” diagnostic testing, e.g. in the ward, outpatients' department, surgery, home, field and workplace. Biosensors to detect one or more biomarkers of the invention include acoustic, plasmon resonance, holographic, Bio-Layer Interferometry (BLI) and microengineered sensors. Imprinted recognition elements, thin film transistor technology, magnetic acoustic resonator devices and other novel acousto-electrical systems may be employed in biosensors for detection of the one or more biomarkers.

Biomarkers for detecting the presence of a disease are essential targets for discovery of novel targets and drug molecules that retard or halt progression of the disorder. As the level of the biomarker is indicative of disorder and of drug response, the biomarker is useful for identification of novel therapeutic compounds in in vitro and/or in vivo assays. Biomarkers described herein can be employed in methods for screening for compounds that modulate the activity of the biomarker.

Thus, in a further aspect of the invention, there is provided the use of a binder or ligand, as described, which can be a peptide, antibody or fragment thereof or aptamer or oligonucleotide directed to a biomarker according to the invention; or the use of a biosensor, or an array, or a kit according to the invention, to identify a substance capable of promoting and/or of suppressing the generation of the biomarker.

The immunoassays described herein include any method employing one or more antibodies or other specific binders directed to bind to the biomarkers defined herein. Immunoassays include 2-site immunoassays or immunometric assays employing enzyme detection methods (for example ELISA), fluorescence labelled immunometric assays, time-resolved fluorescence labelled immunometric assays, chemiluminescent immunometric assays, immunoturbidimetric assays, particulate labelled immunometric assays and immunoradiometric assays as well as single-site immunoassays, reagent limited immunoassays, competitive immunoassay methods including labelled antigen and labelled antibody single antibody immunoassay methods with a variety of label types including radioactive, enzyme, fluorescent, time-resolved fluorescent and particulate labels. All of said immunoassay methods are well known in the art, see for example Salgame et al. (1997) and van Nieuwenhuijze et al. (2003).

Identifying, detecting and/or quantifying can be performed by any method suitable to identify the presence and/or amount of a specific protein in a biological sample from a subject or a purification or extract of a biological sample or a dilution thereof. In particular, quantifying may be performed by measuring the concentration of the target in the sample or samples. Biological samples that may be tested in a method of the invention include those as defined hereinbefore. The samples can be prepared, for example where appropriate diluted or concentrated, and stored in the usual manner. The present invention finds particular use in blood, serum or plasma samples which may be obtained from the subject.

Identification, detection and/or quantification of biomarkers may be performed by detection of the biomarker or of a fragment thereof, e.g. a fragment with C-terminal truncation, or with N-terminal truncation. Fragments are suitably greater than 4 amino acids in length, for example 5, 6, 7, 8, 9, 10, 11, 12, 13, 14, 15, 16, 17, 18, 19, or 20 amino acids in length. It is noted in particular that peptides of the same or related sequence to that of histone tails are particularly useful fragments of histone proteins.

For example, detecting and/or quantifying can be performed by one or more method(s) selected from the group consisting of: SELDI (-TOF), MALDI (-TOF), a 1-D gel-based analysis, a 2-D gel-based analysis, Mass spec (MS), reverse phase (RP) LC, size permeation (gel filtration), ion exchange, affinity, HPLC, UPLC and other LC or LC MS-based techniques. Appropriate LC MS techniques include ICAT® (Applied Biosystems, CA, USA), or iTRAQ® (Applied Biosystems, CA, USA). Liquid chromatography (e.g. high pressure liquid chromatography (HPLC) or low pressure liquid chromatography (LPLC)), thin-layer chromatography, NMR (nuclear magnetic resonance) spectroscopy could also be used.

Methods involving detection and/or quantification of one or more biomarkers of the invention can be performed on bench-top instruments, or can be incorporated onto disposable, diagnostic or monitoring platforms that can be used in a non-laboratory environment, e.g. in the physician's office or at the subject's bedside. Suitable biosensors for performing methods of the invention include “credit” cards with optical or acoustic readers. Biosensors can be configured to allow the data collected to be electronically transmitted to the physician for interpretation and thus can form the basis for e-medicine.

The identification of biomarkers for a disease state permits integration of diagnostic procedures and therapeutic regimes. The biomarkers provide the means to indicate therapeutic response, failure to respond, unfavourable side-effect profile, degree of medication compliance and achievement of adequate serum drug levels. The biomarkers may be used to provide warning of adverse drug response. Biomarkers are useful in development of personalized therapies, as assessment of response can be used to fine-tune dosage, minimise the number of prescribed medications, reduce the delay in attaining effective therapy and avoid adverse drug reactions. Thus by monitoring a biomarker of the invention, subject care can be tailored precisely to match the needs determined by the disorder and the pharmacogenomic profile of the subject, the biomarker can thus be used to titrate the optimal dose, predict a positive therapeutic response and identify those subjects at high risk of severe side effects.

Biomarker-based tests provide a first line assessment of ‘new’ subjects, and provide objective measures for accurate and rapid diagnosis, not achievable using the current measures.

Biomarker monitoring methods, biosensors and kits are also vital as subject monitoring tools, to enable the physician to determine whether relapse is due to worsening of the disorder. If pharmacological treatment is assessed to be inadequate, then therapy can be reinstated or increased; a change in therapy can be given if appropriate. As the biomarkers are sensitive to the state of the disorder, they provide an indication of the impact of drug therapy.

References to “subject”, “individual” or “patient” are used interchangeably herein. The subject may be a human or an animal subject. In one embodiment, the subject is a human. In one embodiment, the subject is a (non-human) animal. In some embodiments the invention encompasses animal subjects (wild or domesticated). In some embodiments, the invention relates to veterinary uses including for livestock and companion animals such as cats, dogs, horses, donkeys, rats, rabbits, mice, guinea pigs, sheep, goats, pigs, deer, llamas, cows and cattle. In one embodiment, the subject is a non-human mammal, such as a dog, mouse, rat or horse, in particular a dog.

The use, panels and methods described herein may be performed in vitro, in vivo or ex vivo. The methods described herein are preferably performed in vitro. References to acts carried out on a body fluid sample “obtained” from a subject are intended to encompass acts carried only a body fluid sample already obtained of “obtainable” from a subject and vice versa.

In one embodiment, the subject is suspected of relapse to a brain tumour. Minimal residual disease (MRD) is the name given to small numbers of cancer cells that remain in the person during treatment, or after treatment when the patient is in remission (i.e. patients with no symptoms or signs of disease). However, MRD is the major cause of relapse in cancer. Methods of the invention are therefore useful in monitoring patients who are suspected of relapse, particularly patients who are in remission from a brain tumour.

The subject tested using the methods described herein may present with symptoms indicative of a brain tumour, for example headaches (caused by pressure in the brain), personality changes, difficulty remembering, trouble speaking or understanding, tiredness, depression, seizures and sight problems.

Detecting and/or quantifying may be compared to a cut-off level. Cut-off values can be predetermined by analysing results from multiple patients and controls, and determining a suitable value for classifying a subject as with or without the disease. For example, for diseases where the level of biomarker is higher in patients suffering from the disease, then if the level detected is higher than the cut-off, the patient is indicated to suffer from the disease. Alternatively, for diseases where the level of biomarker is lower in patients suffering from the disease, then if the level detected is lower than the cut-off, the patient is indicated to suffer from the disease. The advantages of using simple cut-off values include the ease with which clinicians are able to understand the test and the elimination of any need for software or other aids in the interpretation of the test results. Cut-off levels can be determined using methods in the art.

Detecting and/or quantifying may also be compared to a control. It will be clear to those skilled in the art that the control subjects may be selected on a variety of basis which may include, for example, subjects known to be free of the disease or may be subjects with a different disease (for example, for the investigation of differential diagnosis). The “control” may comprise a healthy subject, a non-diseased subject and/or a subject without a brain tumour. Comparison with a control is well known in the field of diagnostics.

Therefore, in one embodiment, the method additionally comprises comparing the level of said cell free nucleosomes in said body fluid sample with one or more controls. For example, the method may comprise comparing the level of cell free nucleosomes present in a body fluid sample obtained from the subject with the level of cell free nucleosomes present in a body fluid sample obtained from a normal subject. The control may be a healthy subject.

Alternatively, the control is a subject with a cancer which is not a particular brain tumour, i.e. the control subject has a different type of cancer or a cancer which affects a different organ in the body or a vascular or haematological cancer.

In one embodiment, the level of cell free nucleosomes is elevated compared to the control.

It will be understood that it is not necessary to measure controls levels for comparative purposes on every occasion. For example, for healthy/non-diseased controls, once the ‘normal range’ is established it can be used as a benchmark for all subsequent tests. A normal range can be established by obtaining samples from multiple control subjects without a particular brain tumour and testing for the level of biomarker. Results (i.e. biomarker levels) for subjects suspected to have a brain tumour can then be examined to see if they fall within, or outside of, the respective normal range. Use of a ‘normal range’ is standard practice for the detection of disease.

In one embodiment, the method additionally comprises determining at least one clinical parameter for the patient. This parameter can be used in the interpretation of results. Clinical parameters may include any relevant clinical information for example, without limitation, gender, weight, Body Mass Index (BMI), smoking status and dietary habits. Therefore, in one embodiment, the clinical parameter is selected from the group consisting of: age, sex and body mass index (BMI).

In one embodiment, the method of the invention is performed to identify a subject at high risk of having a brain tumour and therefore in need of further testing (i.e. further cancer investigations). The further testing may involve one or more of: biopsy, neurological examination, eye tests, diagnostic scans (such as MRI or CT scans).

Methods and biomarkers described herein may be used to identify if a patient is in need of a biopsy. Therefore, according to a further aspect of the invention there is provided a method of identifying a patient in need of a biopsy comprising obtaining a body fluid sample from said patient, detecting the level of cell free nucleosomes in the body fluid sample, and using the results obtained to identify whether the patient is in need of a biopsy.

According to a further aspect of the invention there is provided a method of identifying a patient in need of a biopsy comprising obtaining a body fluid sample from said patient, applying the sample to a panel test as defined herein, and using the results obtained from the panel test to identify whether the patient is in need of a biopsy.

According to a further aspect of the invention, there is provided there is provided the use of binding agents which specifically bind to cell free nucleosomes (in particular, binding agents which specifically bind to H3R8Cit, H3S10Ph and/or H3K4Me2) in the manufacture of a kit for diagnosing and/or monitoring a brain tumour in a body fluid sample.

According to a further aspect of the invention, there is provided there is provided the use of binding agents which specifically bind to H3.1, H3R8Cit, H3S10Ph and/or H3K4Me2 (in particular, binding agents which specifically bind to H3.1, H3R8Cit and/or H3K4Me2) in the manufacture of a kit for diagnosing and/or monitoring a brain tumour in a body fluid sample.

### Additional Biomarkers

The level of cell free nucleosomes may be detected or measured as one of a panel of measurements. The panel may comprise different epigenetic features of the nucleosome as described hereinbefore (e.g. a histone isoform and a PTM).

In one embodiment, the panel comprises one or more cytokines, such as one or more interleukins.

Interleukins (ILs) are a group of cytokines, usually secreted by leukocytes, that act as signal molecules. They have key roles in stimulating immune responses and inflammation. They were first identified in the 1970s and have been designated numerically as more interleukin types have been discovered. Examples of interleukins include, but are not limited to: IL-1, IL-2, IL-3, IL-4, IL-5, IL-6, IL-7, IL-8, IL-9, IL-10, IL-11, IL-12, IL-13, IL-14 and IL-15.

In one embodiment, the one or more interleukins is selected from the group consisting of: Interleukin-6 (IL-6), Interleukin-10 (IL-10) and Interleukin-1β (IL-1β).

The interleukin may be IL-6. Interleukin-6 (IL-6) is a cytokine with a wide variety of biological functions. It is a potent inducer of fever and the acute phase response. The sequence of human IL-6 is known in the art and is described at UniProt Accession No. P05231. In one particular embodiment, the interleukin may be IL-6 and the panel of measurements may comprise measurement of histone isoform H3.1 and IL-6.

Alternatively, or additionally, the interleukin may be IL-10. Interleukin-10 (IL-10) is an anti-inflammatory cytokine with a wide variety of biological functions. The sequence of human IL-10 is known in the art and is described at UniProt Accession No. P22301. In one particular embodiment, the interleukin may be IL-10 and the panel of measurements may comprise measurement of histone post-translational modification H3cit and IL-10.

Alternatively, or additionally, the interleukin may be IL-1β. Interleukin-1β (IL-1β) is a pro-inflammatory cytokine and is involved in a variety of cellular activities, including cell proliferation, differentiation, and apoptosis. In one particular embodiment, the interleukin may be IL-1β and the panel of measurements may comprise measurement of histone isoform H3.1 and IL-1β.

In one embodiment, the panel comprises an epigenetic feature of a cell free nucleosome and an interleukin. In another embodiment, the panel comprises an epigenetic feature of a cell free nucleosome and two interleukins. For example, the cell free nucleosome measurement can be combined with more than one interleukin measurement, such as IL-6 and IL-1β or IL-10 and IL-1β or IL-6 and IL-10. In a further embodiment, the epigenetic feature of a cell free nucleosome is selected from a histone isoform, such as H3.1, and a post translationally modified histone, such as H3cit. In a yet further embodiment, the panel of measurements is H3.1, IL-6 and IL-1β. In an alternative embodiment, the panel of measurements is H3cit, IL-10 and IL-1β.

It will be clear to those skilled in the art, that any combination of the biomarkers disclosed herein may be used in panels and algorithms for the detection of a brain tumour, and that further markers may be added to a panel including these markers.

According to an aspect of the invention there is provided the use of a panel test to detect a patient with a brain tumour, wherein the panel test comprises reagents to detect measurements of nucleosomes or a component thereof and one or more interleukins and/or epigenetic feature(s), in a body fluid sample obtained from the patient.

### Methods of Treatment

According to a further aspect, there is provided a method of treating a brain tumour in a subject, which comprises the following steps:
(i) detecting or measuring the level of cell free nucleosomes in a blood, serum or plasma sample obtained from the subject;
(ii) using the level measured in step (i) as indicative of the presence of said brain tumour in the subject; and
(iii) treating surgically or administering a therapeutic agent if the subject is determined to have said brain tumour in step (ii).
According to a further aspect, there is provided a method of treating a glioblastoma in a subject, which comprises the following steps:
(i) detecting or measuring the level of cell free nucleosomes in a body fluid sample obtained from the subject;
(ii) using the level measured in step (i) as indicative of the presence of said glioblastoma in the subject; and
(iii) treating surgically or administering a therapeutic agent if the subject is determined to have said glioblastoma in step (ii).

According to a further aspect, there is provided a method of treating a brain tumour in a subject in need thereof, which comprises the step of treating surgically or administering a therapeutic agent to a subject identified as having differing levels of cell free nucleosomes in a blood, serum or plasma sample obtained from said subject, when compared to the level of cell free nucleosomes in a blood, serum or plasma sample obtained from a control subject.

According to a further aspect, there is provided a method of treating a glioblastoma in a subject in need thereof, which comprises the step of treating surgically or administering a therapeutic agent to a subject identified as having differing levels of cell free nucleosomes in a body fluid sample obtained from said subject, when compared to the level of cell free nucleosomes in a body fluid sample obtained from a control subject.

In one embodiment, the treatment is selected from one or more of: surgery, chemotherapy, immunotherapy, hormone therapy, biological therapy and radiotherapy. In one embodiment, the treatment is neurosurgery. In one embodiment, the treatment is a combination of chemotherapy and radiotherapy (“chemoradiation”), e.g. comprising radiotherapy over a period of weeks along with rounds of the chemotherapy. In one embodiment, the chemotherapy is selected from temozolomide (TMZ) and carmustine. In one embodiment, the treatment is an immunotherapy, such as bevacizumab (Avastin).

The Methods May Comprise:
(i) measuring the level of cell free nucleosomes (optionally in combination with the level of one or more additional biomarkers) in a body fluid (e.g., blood, serum or plasma) sample obtained from the subject;
(ii) identifying the subject as suffering from a brain tumour based on a higher level of cell free nucleosomes compared to a control; and
(iii) administering a treatment to the subject.

According to another aspect of the invention there is provided a method of treatment for a brain tumour comprising identifying a patient in need of treatment for a brain tumour using a panel test and providing said treatment, wherein the panel test comprises reagents to detect measurements of nucleosomes or a component thereof and one or more additional biomarkers (e.g. as described herein). A patient with a brain tumour is expected to have a higher level of cell free nucleosomes compared to a control.

According to another aspect of the invention there is provided a method of treatment for a glioblastoma comprising identifying a patient in need of treatment for a brain tumour using a panel test and providing said treatment, wherein the panel test comprises reagents to detect measurements of nucleosomes or a component thereof and one or more additional biomarkers (e.g. as described herein). A patient with a brain tumour is expected to have a higher level of cell free nucleosomes compared to a control.

It will be understood that the embodiments described herein may be applied to all aspects of the invention, i.e. the embodiment described for the uses may equally apply to the claimed methods and so forth.

The invention will now be illustrated with reference to the following non-limiting examples.

### EXAMPLES

### EXAMPLE 1: Cell Line Study of Nucleosome-Based Biomarkers in the Diagnosis and Detection of Relapses in Glioblastoma

### Methods

Nucleosome structure was measured using a sandwich immunoassay based on magnetic beads and chemiluminescence technology. 50 μL of diluted nucleosome extract is mixed with an acridinium ester labeled anti-nucleosome detection antibody and incubated for 30 minutes. Then, an anti-histone modification/variant antibody coated beads were added. Finally, trigger solutions were added following a wash step and the signal emitted was measured by the luminometer. The results are expressed in Relative light unit (RLU), the concentration is then evaluated by using a four-parameter logistic regression of a reference standard curve.

### Results

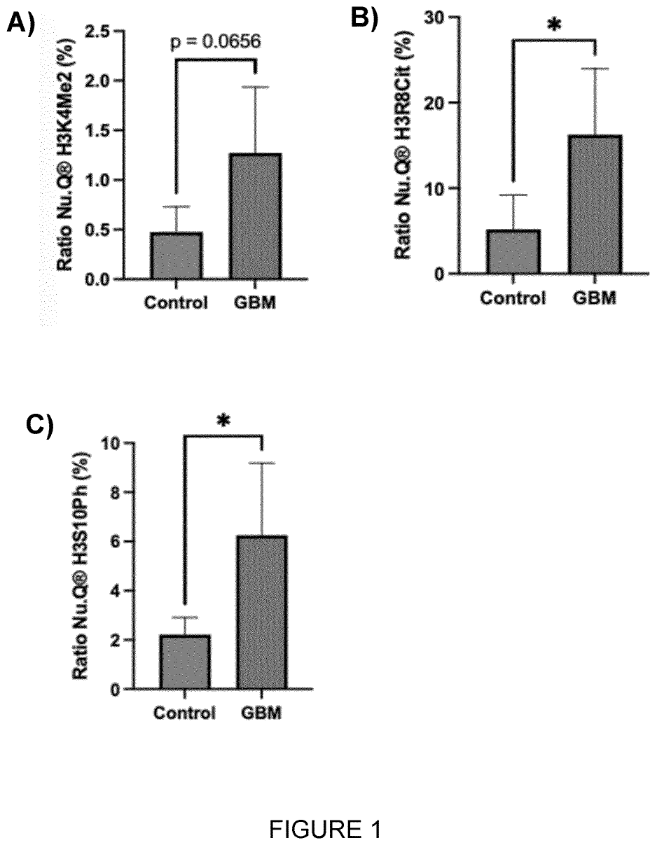

Four glioblastoma cell lines (SF-126, U-87MG, U-118MG, and U-138MG) compared to a healthy microglia cell line (HMC3) and other solid cancer cell lines including pancreas (MIA PaCa-2), liver (HepG2), and cervix/uterus (HeLa) were analysed to identify their epigenetic profile. Nucleosomes were extracted and analysed for 13 histone post-translational modifications (PTMs) as described in the Methods. Quantitative results of PTMs expression were normalized on quantification of total nucleosomes (H3.1-nucleosomes) using an immunoassay Nu.Q® commercially available from Volition. As shown in FIG. 1, the immunoassay identified three candidate biomarkers compared to control cell lines (n=4): citrullinated-histone H3 (H3R8Cit) (control=4.68% ; GBM=13.88% ; p<0.05), phosphorylation of H3S10 (H3S10Ph) (control=2.31% ; GBM=5.57% ; p<0.05), and a trend in H3K4Me2 elevation (control=0.43% ; GBM=1.07% ; p=0.066) in GBM cell lines (n=4).

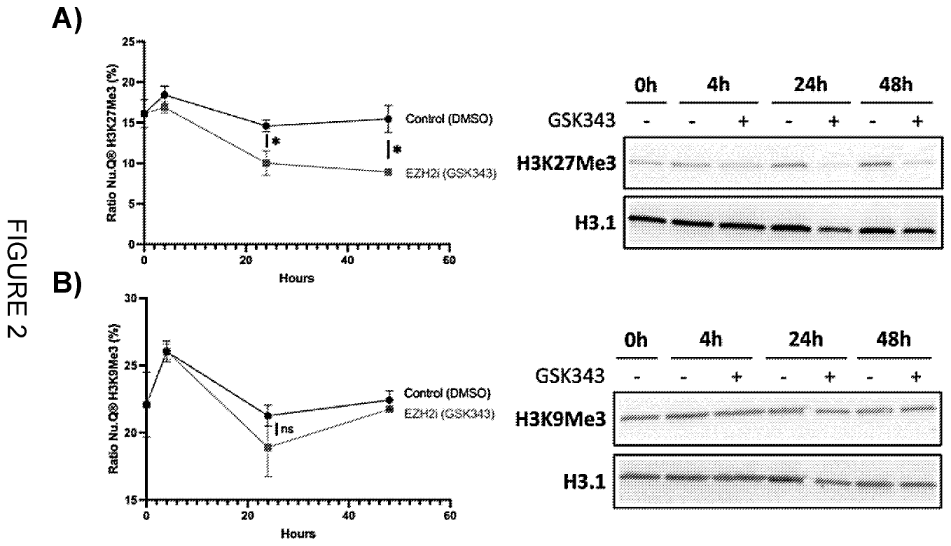

These results were confirmed by western blot. In addition, principal component analysis revealed a segregation between GBM and control cells, mainly driven by these three PTMs. Moreover, to demonstrate the capacity of the nucleosome assays to quantitatively monitor PTMs, GBM cell line SF-126 (n=3) was treated with an EZH2 inhibitor which is responsible of H3K27 methylation) (iEZH2). As shown in FIG. 2, after 24 hours and 48 hours of exposure, the level of expression of H3K27Me3 expressed as ratio of total nucleosomes was decreased by 32% (control=14.67%±0.58%; iEZH2=10.00%±1.73%; p<0.05) and 41% (control=15.33%±1.53%; iEZH2=9.00%±0.00%; p<0.05), respectively. Those results were also confirmed by western blot analysis. The results show we identified three in vitro epigenetic-based marks of GBM and demonstrated that detecting nucleosomes through liquid biopsy is a valuable tool to monitor the degree of PTMs expression.

In summary, the results show that nucleosomes levels, particularly nucleosomes containing H3R8Cit, H3S10Ph and/or H3K4Me2, can be used for brain tumour detection.

### EXAMPLE 2: Study of Nucleosome-Based Biomarkers in the Diagnosis and Detection of Glioblastoma

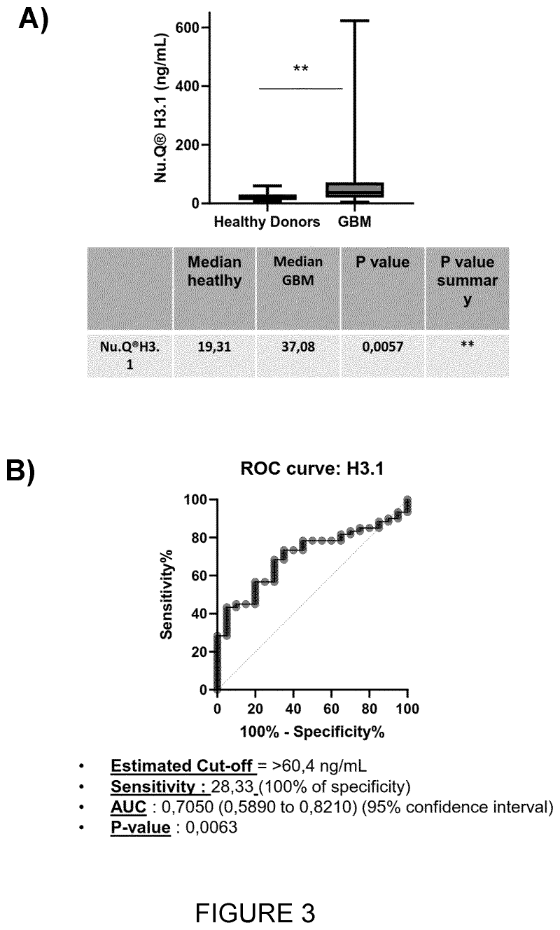

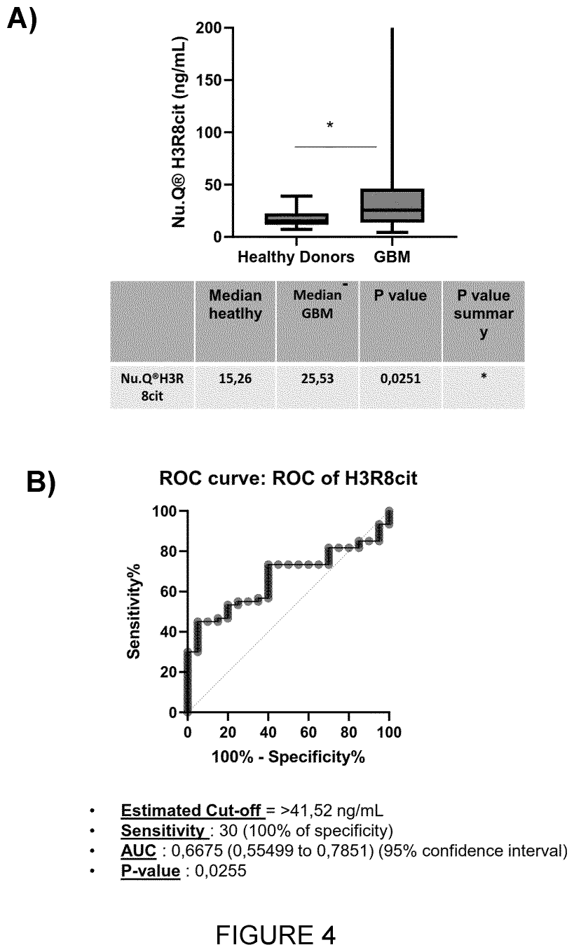

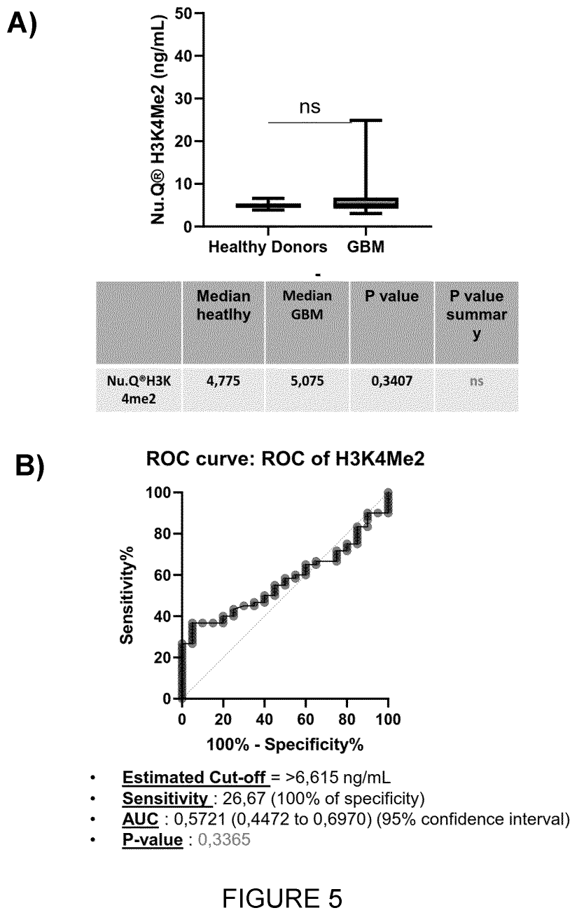

In order to show that methods of the present invention are effective in body fluid samples, plasma taken from GMB patients was compared to plasma from healthy samples. In brief nucleosomes were extracted and analysed for H3.1, H3R8Cit and H3K4Me2 in line with the methods described in Example 1. The results are reported as ng/mL of the candidate biomarker. As shown in the Figures, the immunoassay identified three biomarkers compared to control samples: H3.1 (FIG. 3), H3R8Cit (FIG. 4) and H3K4Me2 (FIG. 5).

We calculated ROC curves and AUCs for the discrimination of subjects with and without GMB for each assay using Logistic Regression analysis. The results are shown in: H3.1 (FIG. 3), H3R8Cit (FIG. 4) and H3K4Me2 (FIG. 5).

In summary, the results show that nucleosomes levels, including nucleosomes containing H3.1, H3R8Cit, and/or H3K4Me2, can be used for brain tumour detection.

1-14. (canceled)

15. A method for diagnosing or detecting a brain tumor, comprising:
(i) contacting a body fluid sample obtained from a subject with a binding agent to detect or measure the level of cell free nucleosomes or a component thereof; and
(ii) using the level of cell free nucleosomes detected to diagnose the subject with the brain tumor.

16. A method for determining the prognosis of a subject with a brain tumor, comprising:
(i) contacting a blood, serum or plasma sample obtained from the subject with a binding agent to detect or measure the level of cell free nucleosomes or a component thereof; and
(ii) using the level of cell free nucleosomes detected as indicative of the prognosis of said brain tumor.

17. A method for monitoring the efficacy of a therapy in a subject having, suspected of having, or being predisposed to a brain tumor, comprising:
(i) contacting a blood, serum or plasma sample obtained from the subject with a binding agent to detect or measure the level of cell free nucleosomes or a component thereof; and
(ii) comparing the level of cell free nucleosomes detected with an earlier blood, serum or plasma sample taken from said subject to determine the efficacy of said therapy.

18. The method of claim 15, wherein the brain tumor is a glioblastoma.

19-20. (canceled)

21. The method of claim 15, wherein the body fluid sample is a blood, serum, plasma or cerebrospinal fluid (CSF) sample.

22. The method of claim 15, wherein the level of cell free nucleosomes or component thereof is detected or measured using an immunoassay, immunochemical, mass spectroscopy, chromatographic, chromatin immunoprecipitation or biosensor method.

23. The method of claim 22, wherein the detection or measurement in step (i) employs a single binding agent or is a 2-site immunometric assay employing two binding agents, or wherein the method of detection or measurement in step (i) comprises contacting the sample with a solid phase comprising a binding agent that detects cell free nucleosomes or a component thereof, and detecting binding to said binding agent.

24. (canceled)

25. The method of claim 23, wherein the binding agent is an antibody.

26. (canceled)

27. The method of claim 15, wherein the method of detection or measurement in step (i) comprises: (a) contacting the sample with a first binding agent which binds to an epigenetic feature of a cell free nucleosome; (b) contacting the sample bound by the first binding agent in step (a) with a second binding agent which binds to cell free nucleosomes; and (c) detecting or quantifying the binding of the second binding agent in the sample.

28. The method of claim 15, wherein the component of the cell free nucleosome comprises an epigenetic feature of the cell free nucleosome.

29. The method of claim 28, wherein the epigenetic feature of the cell free nucleosome is a histone isoform.

30. The method of claim 29, wherein the histone isoform is a core nucleosome.

31. The method of claim 28, wherein the epigenetic feature of the cell free nucleosome is a histone post translational modification (PTM).

32. The method of claim 31, wherein the histone PTM is selected from the group consisting of citrullination, phosphorylation and methylation.

33. The method of claim 31, wherein the histone PTM is selected from the group consisting of H3R8Cit, H3S10Ph, and H3K4Me2.

34-35. (canceled)

36. The method of claim 15, further comprising comparing the level of said cell free nucleosomes in said sample with one or more controls.

37-39. (canceled)

40. A kit comprising one or more reagents for carrying out the method of claim 15, optionally wherein the one or more reagents comprise a reagent to detect one or more of H3.1, H3R8Cit, H3S10Ph and H3K4Me2.

41. (canceled)

42. The method of claim 30, wherein the core nucleosome is a histone H3 isoform.

43. The method of claim 42, wherein the histone H3 isoform is H3.1.
